(19) United States
(12) Patent Application Publication
(10) Pub. No.: US 2004/0092768 A1
(43) Pub. Date: May 13, 2004

(54) Method for the production of acrylic acid or methacrylic acid by gas phase oxidation of propane or isobutane

(76) Inventors: Frieder Borgmeier, Mannheim (DE); Hartmut Hibst, Schriesheim (DE); Andreas Tenten, Maikammer (DE); Wolfgang Mattmann, Limburgerhof (DE); Otto Machhammer, Mannheim (DE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/474,009

(22) PCT Filed: Apr. 3, 2002
(86) PCT No.: PCT/EP02/03690

(30) Foreign Application Priority Data
Apr. 6, 2001 (DE) 10117357.1

(51) Int. Cl. C07C 51/16

(52) U.S. Cl. 562/547; 562/546

(57) ABSTRACT

A process for the preparation of acrylic acid or methacrylic acid is described, in which propane or isobutane is reacted with molecular oxygen in the gas phase in a fluidized-bed reactor containing a catalyst, the catalyst containing a multimetal oxide comprising molybdenum, tellurium and/or antimony, vanadium and niobium, and a catalyst activator which comprises at least one tellurium compound being added to the reactor during the reaction.

The present invention relates to a process for the preparation of acrylic acid or methacrylic acid, in which propane or isobutane is reacted with molecular oxygen in the gas phase over a heterogeneous catalyst, the catalyst comprising a multimetal oxide.

A process for the preparation of acrylic acid or methacrylic acid by gas-phase oxidation of propane or isobutane over a multimetal oxide catalyst is disclosed, for example, in EP-B 608 838, EP-A 895 809, EP-A 962 253, WO 00/29106, WO 98/22421 and JP-A 10-36311. However, the known process is disadvantageous in that the catalytic activity and/or selectivity of the multimetal oxide catalyst deteriorates in the course of time, which leads to a reduction in the yield of the desired unsaturated carboxylic acid.

On the other hand, processes in which a declining activity and/or selectivity of a catalyst is restored by means of a catalyst activator are known. Thus, U.S. Pat. No. 4,709,070 describes a process for the oxidation, ammoxidation or oxidative dehydrogenation of an organic compound in the presence of a tellurium-containing oxide catalyst, a tellurium compound or combination of a tellurium compound and a molybdenum compound being added as a catalyst activator to the reaction system. U.S. Pat. No. 3,882,159 describes a process for the preparation of acrylonitrile or methacrylonitrile by gas-phase ammoxidation of propylene or isobutylene in the presence of a molybdenum-containing oxide catalyst, the ammoxidation being carried out with addition of a catalyst activator in the form of a molybdenum compound to the reaction system. DE 198 36 359 describes a process for the preparation of acrylonitrile or methacrylonitrile by gas-phase ammoxidation of propane or isobutane by means of a mixed oxide catalyst containing molybdenum, tellurium, vanadium and niobium, a catalyst activator in the form of a tellurium compound and, if desired, a molybdenum compound being added to the reaction system. None of these publications discloses a process for the gas-phase oxidation of propane or isobutane to acrylic acid or methacrylic acid.

It is an object of the present invention to provide a process for the preparation of acrylic acid or methacrylic acid by gas-phase oxidation of propane or isobutane, in which a high yield of acrylic acid or methacrylic acid is maintained in a stable manner over a long time span.

We have found that this object is achieved, according to the invention, by a process for the preparation of acrylic acid or methacrylic acid, in which propane or isobutane is reacted with molecular oxygen in the gas phase in a reactor over a heterogeneous catalyst, the catalyst containing a multimetal oxide comprising molybdenum, vanadium and niobium and tellurium and/or antimony, and a catalyst activator which comprises at least one tellurium compound and/or antimony compound is added to the reactor during the reaction.

The catalyst activator used in the novel process comprises at least one tellurium compound and/or at least one antimony compound and, if desired, at least one molybdenum compound. If the molybdenum compound is concomitantly used, it can be fed to the reactor separately from the tellurium and/or antimony compound or together with it.

It is preferable to use a tellurium compound which can be converted into a tellurium oxide under the conditions of the gas-phase oxidation of propane or isobutane. Preferred examples of tellurium compounds include metallic tellurium, inorganic tellurium compounds, such as telluric acid, tellurium dioxide and tellurium trioxide, and organic tellurium compounds, such as methyltellurol, ethyltellurol, propyltellurol and dimethyltellurium oxide, diethyltellurium oxide or dipropyltellurium oxide. Among these, telluric acid is most preferred.

Suitable antimony compounds are metallic antimony, antimony oxides, such as antimony trioxide, antimony tetroxide or antimony pentoxide; hydrated antimony oxides, antimony alkoxides, such as antimony trimethoxide; antimony halides, such as antimony trichloride or antimony pentachloride.

Suitable molybdenum compounds are ammonium heptamolybdate, molybdic acid, molybdenum dioxide and molybdenum trioxide.

The novel process is carried out by bringing a propane or isobutane into contact with a heterogeneous, i.e. solid or preferably particulate, catalyst under conditions under which an oxidation of the propane or isobutane to acrylic acid or methacrylic acid takes place. Suitable reactors, in particular fluidized-bed reactors or fixed-bed reactors, for carrying out the novel process are known to a person skilled in the art.

In the fluidized-bed reactor, starting gas mixture flows through a bed of the finely divided catalyst at a flow rate such that the bed expands with vigorous movement and thorough mixing of the particulate catalyst with the gas phase.

In the fixed-bed reactor, the catalyst is arranged in a manner such that it is stationary while the starting gas mixture is flowing through. As a rule, the catalyst is introduced into a plurality of tubes which are arranged parallel and through which the starting gas mixture flows and which are surrounded by a heat exchange medium for removing the heat of reaction.

Suitable as propane or isobutane which are used in the novel process are the corresponding gases having a purity as available on the industrial scale. A suitable source of the molecular oxygen is in particular air, air enriched with oxygen or pure oxygen. If required, an inert gas, such as helium, argon, nitrogen, carbon dioxide, steam or the like can be concomitantly used. The molar ratio of propane or isobutane to the molecular oxygen is in general 1:0.2-10, preferably 1:0.5-5. The gas-phase oxidation temperature is in general from 300 to 500° C., preferably from 350 to 470° C. The gas pressure is in general from 0.5 to 10, preferably from 0.8 to 5, bar. The residence time of the gaseous starting material in the reactor is in general from 0.5 to 20, preferably from 1 to 10, seconds.

The present invention is not subject to any particular restrictions with regard to the method of addition of the catalyst activator to the reactor. The activator can be added to the reactor separately or together with the starting gas mixture. When a fluidized-bed reactor is used, the separate addition is expediently effected via a pipeline directly into the fluidized bed of the reactor in which the catalyst is present in high concentration. This method of addition permits sufficient contact between the activator and the catalyst. When a fixed-bed reactor is used, the catalyst activator is preferably added to the stream of the starting gas mixture. When a fluidized-bed reactor is used, the catalyst activator is preferably present in particulate form and comprises particles having a size of more than 10 μm, in particular from 25 μm to 1 mm. The vigorous movement and thorough mixing of the catalyst particles in the fluidized-bed reactor permit good interaction with the activator particles. When a fixed-bed reactor is used, the catalyst activator is preferably volatile or sublimable or is present as particles having a size of less than 500 μm, in particular less than 250 μm, particularly preferably less than 150 μm. In this way, it is ensured that the activator is uniformly distributed in the reactor by the flowing starting gas mixture.

The catalyst activator can be added continuously or periodically. Regarding the frequency of the addition of the activator and the amount of the activator fed to the reactor, the present invention is not subject to any particular restrictions. The frequency and amount can readily be determined by a person skilled in the art on the basis of simple experiments, by adding varying amounts of activator to the reactor and monitoring the results of the gas-phase oxidation. The amounts of an activator portion fed in is preferably from 0.01 to 20, particularly preferably up to 10, % by weight, expressed as the amount of tellurium and/or antimony, based on the original amount of tellurium and/or antimony which is contained in the catalyst load of the fluidized-bed reactor.

If the activator contains a molybdenum compound in addition to the tellurium compound, the amount of the molybdenum compound in an activator portion is preferably from 0.01 to 10, in particular up to 5, % by weight, expressed as the amount of molybdenum, based on the original amount of molybdenum which is contained in the catalyst load in the fluidized-bed reactor.

The type of interaction between catalyst and catalyst activator, by means of which the catalyst activity and/or catalyst selectivity are regenerated, has not been completely explained. Presumably, the activator or components or decomposition products thereof can diffuse or sublime into the multimetal oxide phase of the catalyst and thus at least partly restore a damaged crystal structure of the multimetal oxide phase.

Any catalyst known per se and having an active phase of a multimetal oxide containing molybdenum, tellurium/antimony, vanadium and niobium is suitable for carrying out the novel process. The catalyst may be supported or unsupported but is preferably supported. A preferred support is a silicon dioxide support. Other support materials which may be used are aluminas, titanium dioxide, zirconium dioxide and mixed oxides thereof with silicon dioxide. Supported catalysts are suitable in particular for use in fluidized-bed reactors. For use in fixed-bed reactors, the catalyst is preferably present as an unsupported catalyst or as a coated catalyst.

Preferably, the multimetal oxide phase of the catalyst used has the following formula:

Mo₁YₐV_{b}Nb_{c}X_{d}Oₙ

where

Y is at least one element selected from tellurium and antimony,

X is at least one element selected from tantalum, tungsten, chromium, titanium, zirconium, bismuth, tin, hafnium, manganese, iron, ruthenium, cobalt, rhodium, nickel, palladium, platinum, zinc, aluminum, gallium, indium, thallium, phosphorus and the alkaline earth metals;

a is from 0.01 to 1.0; preferably from 0.05 to 0.5;

b is from 0.01 to 1.0; preferably from 0.1 to 0.5;

c is from 0.01 to 1.0; preferably from 0.05 to 0.5;

d is from 0 to 1.0, preferably from 0.01 to 0.5, and

n is a number which is determined by the valency and frequency of the elements other than oxygen in the multimetal oxide.

For the preparation of suitable catalysts, sources of the elements other than oxygen which constitute the multimetal oxide are usually thoroughly mixed and, if required, dried and calcined. The thorough mixing can be effected in dry or wet form. Suitable sources of the elements other than oxygen which constitute the multimetal oxide are oxides or compounds which can be converted into oxides by heating, if required in the presence of oxygen, such as nitrates, oxalates, acetates, hydroxides, carbonates, bicarbonates and ammonium salts of element-oxygen compounds. Preferred examples are ammonium heptamolybdate [(NH₄)₆Mo₇O₂₄×4H₂O] as a molybdenum source; telluric acid (H₆TeO₆) as a tellurium source, antimony trioxide or antimony tetroxide as an antimony source, ammonium metavanadate (NH₄VO₃) as a vanadium source; ammonium niobium oxalate and niobic acid (Nb₂O₅×nH₂O) as a niobium source.

If a silicon dioxide-supported catalyst is desired, a silica sol is preferably concomitantly used as a silicon dioxide source. Silica sols which are ammonium-stabilized are preferably used for this purpose.

For the catalyst preparation, the sources of the elements other than oxygen which constitute the multimetal oxide are preferably dissolved or suspended in an aqueous phase; the aqueous solution or suspension is dried, the resulting catalyst precursor material being calcined to give the active catalyst, if necessary after shaping. Alternatively, the catalyst precursor material or the calcined multimetal oxide can be applied to molded catalyst supports.

For the preparation of a preferred catalyst, a first aqueous solution is initially prepared by dissolving ammonium heptamolybdate, telluric acid and ammonium metavanadate in water. Separately therefrom, ammonium niobium oxalate or oxalic acid and niobic acid are dissolved in water, a second aqueous solution being obtained. A nitrate, oxalate, acetate, hydroxide, oxide, carbonate or bicarbonate of an element selected from tantalum, tungsten, chromium, titanium, zirconium, antimony, bismuth, tin, hafnium, manganese, iron, ruthenium, cobalt, rhodium, nickel, palladium, platinum, zinc, aluminum, gallium, indium, thallium, phosphorus and the alkaline earth metals or an ammonium salt of an oxygen compound of such an element is dissolved in water separately therefrom, a third aqueous solution being obtained.

The second and third aqueous solutions are added in succession to the first aqueous solution and, if required, a silica sol is added. The sequence of the addition can be changed as desired.

The combined aqueous solutions are then dried, preferably by spray-drying. The spray-drying is carried out by a conventional method, for example by means of a binary nozzle, a high-pressure nozzle or a centrifugal method, a dried, particulate catalyst precursor material being obtained. Preheated air is preferably used for the spray-drying; the inlet temperature at the spray dryer is suitably from 150 to 350° C.

It is preferable to choose the droplet size in the spray-drying so that the catalyst obtained after calcination has a particle diameter of from 5 to 120 μm and preferably a mean particle diameter of from 25 to 70 μm.

The dried, particulate catalyst precursor is then calcined. The calcination can be carried out in an oxidizing, reducing or inert atmosphere. It is preferably effected in an inert gas atmosphere, such as nitrogen, argon or helium, which is substantially free of oxygen. The temperature of the calcination is as a rule from 500 to 700° C., preferably from 550 to 650° C. The duration of the calcination is usually from 0.5 to 20, preferably from 1 to 8, hours. Conventional furnaces, such as rotary kilns, tunnel furnaces, muffle furnaces or fluidized-bed furnaces, are suitable for the calcination. Before the calcination, the dried catalyst precursor material can be heat-treated in an oxygen-containing atmosphere, for example air, at from 200 to 400° C. for from 1 to 5 hours.

For the preparation of coated catalysts, the active material is applied to inert catalyst supports, it being possible to effect the application before or after the final calcination. As a rule, the relevant material is calcined before the supports are coated.

The coating of the supports for the preparation of the coated catalysts is carried out as a rule in a suitable rotatable container. Expediently, the powder material to be applied can be moistened and can be dried again, for example by means of hot air, after the application. The coat thickness of the powder material applied to the support is expediently chosen to be from 50 to 500 μm, preferably from 150 to 250 μm. The powder material can also advantageously be applied to the supports from a suspension, for example by spraying the dispersion onto the moving supports or simultaneously passing over an inert gas.

Conventional porous or nonporous aluminas, silicon dioxide, thorium dioxide, zirconium dioxide, silicon carbide or silicates, such as magnesium silicate or aluminum silicate, can be used as support materials. The supports may have a regular or irregular shape, those having a regular shape and substantial surface roughness, for example spheres or hollow cylinders, being preferred. The use of substantially nonporous steatite rings having a rough surface is particularly advantageous.

For the preparation of unsupported catalysts, the precursor material is compacted, before or after the calcination, to give the desired catalyst geometry (for example by pelleting or extrusion), it being possible, if required, to add the customary assistants, for example graphite or stearic acid, as lubricants and/or molding assistants, and reinforcing agents, such as microfibers of glass, asbestos, silicon carbide or potassium titanate. Preferred geometries for unsupported catalysts are hollow cylinders having an external diameter and a length of from 2 to 10 mm and a wall thickness of from 1 to 3 mm.

We claim:

1. A process for the preparation of acrylic acid or methacrylic acid, in which propane or isobutane is reacted with molecular oxygen in the gas phase in a reactor over a heterogeneous catalyst, the catalyst containing a multimetal oxide comprising molybdenum, vanadium and niobium and tellurium and/or antimony, and a catalyst activator which comprises at least one tellurium compound and/or antimony compound is added to the reactor during the reaction.

2. A process as claimed in claim 1, in which the catalyst activator additionally comprises at least one molybdenum compound.

3. A process as claimed in claim 1 or 2, in which the catalyst comprises a silicon dioxide support.

4. A process as claimed in claim 1 or 2, in which the catalyst is an unsupported catalyst or a coated catalyst.

5. A process as claimed in any of the preceding claims, in which the multimetal oxide has the following formula:
Mo₁YₐV_{b}Nb_{c}X_{d}Oₙ
where
Y is at least one element selected from tellurium and antimony,
X is at least one element selected from tantalum, tungsten, chromium, titanium, zirconium, bismuth, tin, hafnium, manganese, iron, ruthenium, cobalt, rhodium, nickel, palladium, platinum, zinc, aluminum, gallium, indium, thallium, phosphorus and the alkaline earth metals;
a is from 0.01 to 1.0;
b is from 0.01 to 1.0;
c is from 0.01 to 1.0;
d is from 0 to 1.0 and
n is a number which is determined by the valency and frequency of the elements other than oxygen in the multimetal oxide.

6. A process as claimed in any of the preceding claims, in which the tellurium compound is selected from the metallic tellurium, inorganic tellurium compounds and organic tellurium compounds.

7. A process as claimed in claim 6, in which the tellurium compound is telluric acid.

8. A process as claimed in any of the preceding claims, in which the antimony compound is selected from metallic antimony, is antimony oxides, hydrated antimony oxides, antimony alkoxides and antimony halides.

9. A process as claimed in any of claims 2 to 8, in which the molybdenum compound is selected from ammonium heptamolybdate, molybdic acid, molybdenum dioxide and molybdenum trioxide.

10. A process as claimed in any of the preceding claims, in which the reactor is a fluidized-bed reactor.

11. A process as claimed in claim 10, in which the catalyst activator is present in particulate form and comprises particles having a size of more than 10 μm.

12. A process as claimed in any of claims 1 to 9, in which the reactor is a fixed-bed reactor.

13. A process as claimed in claim 12, in which the catalyst activator is volatile or sublimable or is present in particulate form as particles having the size of less than 500 μm.
